(19) United States
(12) Patent Application Publication
(10) Pub. No.: US 2023/0181759 A1
(43) Pub. Date: Jun. 15, 2023

(54) COMPOSITIONS FOR TREATMENT OF VIRAL RESPIRATORY INFECTIONS AND METHODS OF USE THEREOF

(71) Applicant: Washington University, St. Louis, MO (US)

(72) Inventors: ARMIN GHOBADI, St. Louis, MO (US); ABDEL KAREEM AZAB, St. Louis, MO (US)

(21) Appl. No.: 17/916,744

(22) PCT Filed: Apr. 1, 2021
(86) PCT No.: PCT/US2021/025437

§ 371 Date: Oct. 3, 2022

Related U.S. Application Data
(60) Provisional application No. 63/003,454, filed on Apr. 1, 2020.

(51) Int. Cl. A61K 47/69; A61K 47/68; A61P 31/14; A61K 31/675

(52) U.S. Cl. CPC A61K 47/6913; A61K 47/6871; A61P 31/14; A61K 31/675

(57) ABSTRACT

Compositions and methods for treating a viral infection may comprise use of a nanoparticle composition. A nanoparticle composition of the present disclosure may comprise a targeting moiety and/or anti-viral agent and reduces the infectivity of a virus for a host cell. A method of treating a viral infection may comprise administering a composition comprising a nanoparticle of the present disclosure, to a subject and reducing the infectivity of the virus for a host cell of the subject. The compositions may be administered via intranasal or systemic administration to treat or prevent a viral infection, for example a coronavirus infection.

### CROSS REFERENCE TO RELATED APPLICATION

This application claims the benefit of U.S. Provisional Application No. 63/003,454, filed Apr. 1, 2020 the disclosure of which is herein incorporated by reference in its entirety.

### FIELD OF THE TECHNOLOGY

The present disclosure encompasses compositions for enhanced therapy for respiratory viral infections and methods of use thereof. In particular, the disclosure relates to compositions and methods to improve the local delivery to the lung while reducing systemic side effects of therapeutic agents for treating respiratory viruses, such as coronaviruses.

### BACKGROUND

Viral infections are responsible for hundreds of thousands of deaths each year. However, treatment options are limited for many viruses. Additionally, carriers of a virus may be asymptomatic, leading to high transmission rates from infected but asymptomatic individuals. There is a need for improved drugs to treat viral infections in both symptomatic and asymptomatic individuals. Furthermore, people such as healthcare workers who are in contact with infected individuals are at high-risk of infection. There is a need for drugs to prevent viral infections in at-risk individuals and other members of the population.

### SUMMARY

Among the various aspects of the present disclosure provide compositions comprising an effective amount of a nanoparticle composition disclosed herein and methods of use thereof.

In an aspect of the disclosure provides method for treating method of reducing or treating a respiratory viral infection in a subject by administering to the subject an effective amount of a nanoparticle composition where the nanoparticle has at least one targeting moiety conjugated to the surface, where the targeting moiety is selected from a recombinant angiotensin converting enzyme-2 (ACE-2) polypeptide and an anti-ACE-2 antibody. In one aspect, the nanoparticle is a liposome and may also include an anti-viral agent.

In a further aspect the disclosure provides pharmaceutical compositions comprising at least one pharmaceutically acceptable excipient and a nanoparticle comprising at least one targeting moiety conjugated to the surface, wherein the targeting moiety is a recombinant angiotensin converting enzyme-2 (ACE-2) polypeptide. In one aspect, the nanoparticle is a liposome and may also include an anti-viral agent. In some embodiments, the pharmaceutical composition is formulated for nasal delivery.

In still a further aspect the disclosure provides pharmaceutical composition comprising at least one pharmaceutically acceptable excipient and a nanoparticle comprising at least one targeting moiety conjugated to the surface, wherein the targeting moiety is an anti-ACE-2 antibody. In one aspect, the nanoparticle is a liposome and may also include an anti-viral agent. In some embodiments, the pharmaceutical composition is formulated for nasal delivery.

While multiple embodiments are disclosed, still other embodiments of the present disclosure will become apparent to those skilled in the art from the following detailed description, which shows and describes illustrative embodiments of the invention.

### BRIEF DESCRIPTION OF THE FIGURES

Those of skill in the art will understand that the drawings, described below, are for illustrative purposes only. The drawings are not intended to limit the scope of the present teachings in any way.

The application file contains at least one drawing executed in color. Copies of this patent application publication with color drawing(s) will be provided by the Office upon request and payment of the necessary fee.

FIG. 1A-1B depict exemplary liposomal preparation and their characterization. FIG. 1A shows a workflow for producing liposomes containing an anti-viral agent made using the thin film hydration method and further conjugated with either anti-ACE2 antibody (a-ACE2) or recombinant human ACE2 protein (r-ACE2). FIG. 1B shows the formulation, size, polydispersity index and zeta potential of the liposome formulations.

FIG. 2A-2B depict the in vivo biodistribution of exemplary liposomal formulations. FIG. 2A shows the biodistribution of intraperitoneal administered liposome formulations. FIG. 2B shows the biodistribution of intranasally administered liposome formulations.

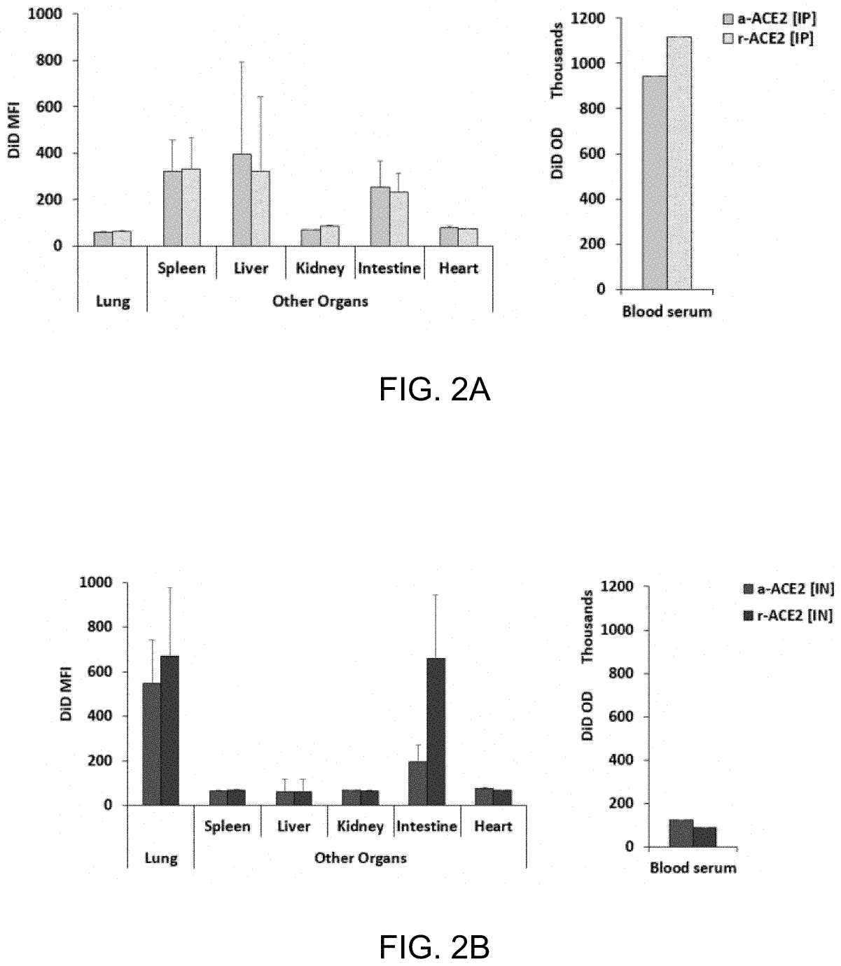

### DETAILED DESCRIPTION

The present disclosure is based, at least in part, on the discovery that the nanoparticle compositions (e.g., liposome composition) according to the disclosure function to bind the virus in vivo thereby reducing the amount of virus capable to infect the host cells and at the same time the nanoparticle compositions deliver an anti-viral agent by specifically targeting cells and tissues susceptible to infection by a respiratory virus (e.g., a coronavirus). In addition, the nanoparticle compositions according to the disclosure are useful to increase angiotensin converting enzyme-2 (ACE-2) levels in cells and tissues susceptible to infection by a respiratory virus. Increasing ACE-2 levels using a nanoparticle composition as described herein can prevent and/or resolve acute lung injury in subjects with infection. In some embodiments, the present disclosure provides nanoparticle compositions which functions as a nanoparticle delivery system of anti-viral drugs resulting in more stability, less toxicity, and targeted delivery of the anti-viral drug loaded.

Severe acute respiratory syndrome coronavirus 1 (SARS-CoV-1) that was responsible for SARS epidemic in 2002-2004, Middle East respiratory syndrome coronavirus (MERS-CoV) that caused MERS first reported in 2012, and SARS-CoV-2 that has been responsible for the more recent coronavirus disease 2019 (Covid-19) pandemic all bind to angiotensin converting enzyme 2 (ACE2) on the surface of the cells in order to infect the cells. Basically ACE-2 is the functional receptor for SARS-CoV-1, SARS-CoV-2, and MERS-COV and most likely future SARS-COV variants. ACE-2 is an important component of Renin-Angiotensin-Aldosterone System (RAAS). ACE-2 converts angiotensin 2 to angiotensin 1-7. High angiotensin 2 is associated with vasoconstriction, inflammation, and acute lung injury. ACE2 is expressed in various organs including lungs, heart, kidney, liver, intestine, and other tissues.

SARS-CoV virus bind to ACE-2 and enter the cells and at the same time downregulate ACE-2 in the surface of the cells of the infected tissue. ACE-2 downregulation results in increased angiotensin 2 that plays an important role in the acute lung injury that is the main cause of death in patients infected with SARS-CoV-2 and similar SARS-CoV variants.

Disclosed herein are compositions, methods, and treatment plans for treating an individual who is at risk of having a respiratory viral infection, has mild symptoms of a respiratory viral infection, or has severe symptoms of a respiratory viral infection. A composition of the present disclosure comprising a nanoparticle composition disclosed herein may be used to treat, prevent, or reduce the infectivity of a respiratory viral infection. A treatment plan may comprise administering a composition (e.g., a composition comprising a liposome composition of the disclosure) to an individual at risk of having a viral infection or who has a viral infection, thereby preventing or treating the viral infection. In some embodiments, a viral infection may be prevented by reducing the amount of virus capable of binding to a host cell or tissue. For example, a composition of the present disclosure may comprise a nanoparticle conjugated to a host viral receptor or fragment thereof (e.g., ACE-2). In some embodiments, a host viral receptor or fragment thereof binds to the virus and at the same time targets the nanoparticle to infected host cells and tissues. In some embodiments, a viral infection may be prevented by disrupting interactions between a viral surface proteins and host cell proteins that activate or enhance insertion of the viral genetic material into the host cell. For example, interactions between a SARS-CoV-2 spike protein, and a host cell ACE-2 receptor.

A composition of the present disclosure may be formulated for locally, for example intra-nasally (e.g., as a nasal spray, or inhalation), or systemically (e.g., intravenous or intraperitoneal) and administered for treating or preventing a respiratory viral infection (e.g., a coronavirus infection such as SARS-CoV-2). The compositions of the present disclosure (e.g., compositions formulated for nasal delivery or inhalation) may be administered to a subject who may be at risk of contracting a viral infection (e.g., SARS-CoV-2). For example, the compositions of the present disclosure may be administered to individuals in high risk environments (e.g., healthcare workers), individuals who have been or who are suspected to have been exposed to a virus (e.g., SARS-CoV-2), or individuals who have tested positive for a viral infection. A composition of the present disclosure may be administered to an individual who is displaying symptoms of a respiratory infection (e.g., a SARS-CoV-2 infection) or who is asymptomatic at the time of administration. In some embodiments, the compositions of the present disclosure may be self-administered by the individual (e.g., as a nasal spray or inhalation) and may be administered outside of a medical facility (e.g., at home).

In some embodiments, the methods and compositions provided herein may prevent or reduce the infectivity of a viral infection by preventing internalization of a virus into a cell of the subject or by preventing internalization of a viral genome into a cell of the subject. In some embodiments, a composition provided herein may disrupt or prevent an interaction between a viral surface protein (e.g., a spike protein or an envelope protein) and a host receptor protein (e.g., an epithelial angiotensin converting enzyme (ACE)). For example, a nanoparticle composition may block internalization of a coronavirus into a cell of a subject by blocking or disrupting interactions between a coronavirus spike protein and a host receptor protein or sequestering the virus in vivo allowing for the virus bound to the nanoparticle composition to be eliminated by immune cells. Administering a nanoparticle composition to a subject at risk for a viral infection may reduce the risk of coronavirus infection in the subject. A composition to treat or prevent a viral infection may comprise a nanoparticle composition. In some embodiments, the nanoparticle composition may comprise an anti-viral agent.

The methods and compositions disclosed herein may be used to treat, prevent, or reduce the infectivity of a respiratory viral infection. In some embodiments, the viral infection may be a coronavirus infection. Pathogens with long incubation periods, such as SARS-CoV-2 which has a median incubation period of about five days, may have high risk of transmission since many infected individuals may be unaware that they are infected. Additionally, carriers of coronavirus may frequently be asymptomatic or have mild symptoms, leading to unknowing contact between a viral host and other members of a population. A subject at risk for a coronavirus infection may come in contact with an asymptomatic carrier of the coronavirus infection, thereby unknowingly contracting the coronavirus infection. Methods and compositions are needed to prevent coronavirus infections in at-risk individuals (e.g., individuals who have come in contact with a carrier of a coronavirus or who may come in contact with a carrier of a coronavirus).

In some embodiments, the compositions, methods, or treatment regiments disclosed herein may treat or prevent a SARS-CoV-2 infection (e.g., COVID-19). A SARS-CoV-2 infection may depend on host cell ACE-2 enzyme. In some embodiments, a SARS-CoV-2 infection may be blocked (e.g., prevented, treated, or slowed) by a nanoparticle composition of the disclosure. Cell entry of a coronavirus (e.g., SARS-CoV-2) may depend on binding of the viral spike (S) proteins to cellular receptors and on S protein priming by host cell proteases. For example, SARS-CoV-2 may use an ACE-2 receptor for entry. A composition of the present disclosure to treat or prevent coronavirus infection may comprise a nanoparticle comprising a host viral receptor conjugated to the nanoparticle surface or a nanoparticle comprising an antibody which specifically binds a host viral receptor conjugated to the nanoparticle surface. In some embodiments, the nanoparticle contains an anti-viral agent (e.g., remdesivir, chloroquine, hydroxychloroquine, lopinavir, ranitidine bismuth citrate, and ritonavir).

Discussed below are components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules of the compound are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Various aspects of the invention are described in further detail in the following sections.

### I. COMPOSITIONS

A composition of the present disclosure may comprise one or more active agents. In some embodiments, an active agent may be an agent to prevent, treat, or reduce the infectivity of a viral infection. In some embodiments, treating a viral infection may comprise reducing the infectivity of the virus. In some embodiments, preventing a viral infection may comprise reducing the infectivity of the virus. A composition of the present disclosure may comprise an active agent to prevent a viral infection, an active agent to treat a viral infection, an active agent to reduce the infectivity of a viral infection, or a combination thereof. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents, or antioxidants.

The present disclosure relates to compositions of a nanoparticle composition and methods of using a nanoparticle composition to treat or prevent a respiratory viral infection. A nanoparticle composition of the disclosure may comprise liposome nanoparticles that encapsulate an anti-viral agent that is specifically delivered to cells susceptible to infection by the virus. The surface of the liposome nanoparticles can conjugated to a recombinant host cell viral receptor or specific antibodies that recognize and bind to a host cell viral receptor on the surface of target cells. Nanoparticles that are bound specifically to the virus or target cells may treat or prevent a viral infection.

Other aspects of the invention are described in further detail below.

### a) Nanoparticle Composition

The present disclosure provides for a nanoparticle composition. In some embodiments, the composition comprises nanoparticles that have at least one targeting molecule conjugated to the surface of the nanoparticles. In some embodiments, the composition comprises nanoparticles that have at least one anti-viral agent encapsulated in the nanoparticles. A composition of the present disclosure may also comprise a suitable pharmaceutically acceptable carrier known in the art. As used herein, the term nanoparticle refers to a particle that has a diameter of less than 1 um (1000 nm). Nanoparticles may be substantially spherical in shape and the diameter of a group of nanoparticles may be represented by the average diameter of the nanoparticles in the group.

Nanoparticles of the present disclosure may be constructed by a variety of materials. Non-limiting examples of the materials a nanoparticle may be constructed from may include polymers, lipids, inorganic substances, and biological materials. In an aspect, a nanoparticle of the present disclosure may be constructed of lipids. In an exemplary embodiment, a nanoparticle of the disclosure is a liposome.

According to the present disclosure, the liposomes contained in the nanoparticle composition can be any liposome and are not particularly limited. In general, the liposomes of the present disclosure can have any liposome structure, e.g., structures having an inner space sequestered from the outer medium by one or more lipid bilayers, or any microcapsule that has a semi-permeable membrane with a lipophilic central part where the membrane sequesters an interior. A lipid bilayer can be any arrangement of amphiphilic molecules characterized by a hydrophilic part (hydrophilic moiety) and a hydrophobic part (hydrophobic moiety). Usually amphiphilic molecules in a bilayer are arranged into two dimensional sheets in which hydrophobic moieties are oriented inward the sheet while hydrophilic moieties are oriented outward. Amphiphilic molecules forming the liposomes of the present invention can be any known or later discovered amphiphilic molecules, e.g., lipids of synthetic or natural origin or biocompatible lipids. Liposomes of the present disclosure can also be formed by amphiphilic polymers and surfactants, e.g., polymerosomes and niosomes. For the purpose of this disclosure, without limitation, these liposome-forming materials also are referred to as “lipids”.

Generally speaking, liposomes are spherical vesicles with a phospholipid bilayer membrane. The lipid bilayer of a liposome may fuse with other bilayers (e.g., the cell membrane), thus delivering the contents of the liposome to cells. Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palm itoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes may be prepared by any known method of preparing liposomes for drug delivery, such as, for example, detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of an anti-viral agent, concentration and composition of lipid, concentration of multivalent cations, rate of mixing, presence of and concentration of solvent. Examples of methods suitable for making liposome composition of the present disclosure include extrusion, reverse phase evaporation, sonication, solvent (e.g., ethanol) injection, microfluidization, detergent dialysis, ether injection, and dehydration/rehydration. The size of liposomes can be controlled by controlling the pore size of membranes used for low pressure extrusions or the pressure and number of passes utilized in microfluidisation or any other suitable methods. In one embodiment, the desired lipids are first hydrated by thin-film hydration or by ethanol injection and subsequently sized by extrusion through membranes of a defined pore size; most commonly 0.05 μm, 0.08 μm, or 0.1 μm.

In general, a variety of lipid components can be used to make the liposomes of the present disclosure. Lipid components usually include, but are not limited to (1) uncharged lipid components, e.g., cholesterol, ceramide, diacylglycerol, acyl(poly ethers) or alkylpoly(ethers); (2) neutral phospholipids, e.g., diacylphosphatidylcholines, sphingomyelins, and diacylphosphatidylethanolamines, (3) anionic lipids, e.g., diacylphosphatidylserine, diacylphosphatidylglycerol, diacylphosphatidate, cardiolipin, diacylphosphatidylinositol, diacylglycerolhemisuccinate, diaclyglycerolhemigluratate, cholesterylhemi succinate, cholesterylhemiglutarate, and the like; (4) polymer-conjugated lipids, e.g., N-[methoxy-(poly(ethylene glycol)diacylphosphatidylethanolamine, poly(ethylene glycol)-diacylglycerol, poly(ethylene glycol)-ceramide; and (5) cationic lipids, e.g., 1,2,-diacyl-3-trimethylammonium-propane (DOTAP), dimethyldioctadecylammonium bromide (DDAB), and 1,2-diacyl-sn-glycero-3-ethylphosphocholine. Monoacyl-substituted derivatives of these lipids, as well as di- and monoalkyl-analogs can be also employed.

Various lipid components can be selected to fulfill, modify or impart one or more desired functions. For example, phospholipid can be used as principal vesicle-forming lipid. Inclusion of cholesterol is useful for maintaining membrane rigidity and decreasing drug leakage. Polymer-conjugated lipids can be used in the liposomal formulation to increase the lifetime of circulation via reducing liposome clearance by liver and spleen, or to improve the stability of liposomes against aggregation during storage, in the absence of circulation extending effect. While inclusion of PEG-lipids in the amount 1 mol % or above of the liposome lipid is asserted to have a several-fold prolongation of the liposome blood circulation time (see, e.g., U.S. Pat. No. 5,013,556).

Nanoparticle compositions containing an anti-viral agent can be made by any suitable methods, e.g., formation of liposomes in the presence of the anti-viral agent. The substituted ammonium and/or polyanion outside of the liposomes can be removed or diluted either following liposome formation or before loading or entrapping a desired entity. Alternatively, liposome composition containing the anti-viral agent can be made via ion exchange method directly or via an intermediate free acid step having a gradient of the anti-viral agent. The anti-viral agent may be associated with the surface of, encapsulated within, surrounded by, or dispersed throughout the nanoparticle. Non-limiting examples of an anti-viral agent include remdesivir, chloroquine, hydroxychloroquine, lopinavir, ranitidine bismuth citrate, and ritonavir.

A nanoparticle of the present disclosure may release an anti-viral agent inside a cell of interest or at a site of interest. In an aspect, a nanoparticle may have controlled release properties, that is, be able to release an anti-viral agent inside a cell of interest or at a site of interest over a period of time. In some aspects, disclosed nanoparticles may substantially immediately release the active agent, in the cell or site of interest.

According to the present disclosure, the nanoparticle contained in the nanoparticle composition of the present disclosure can also be a targeting nanoparticle, e.g., a nanoparticle containing one or more targeting moieties or biodistribution modifiers on the surface of the nanoparticle. A targeting moiety can be any agent that is capable of specifically binding or interacting with a desired target. A targeting moiety may be attached to the surface of a nanoparticle through covalent, non-covalent, or other associations. Non-limiting examples of targeting moieties may include synthetic compounds, natural compounds or products, macromolecular entities, and bioengineered molecules, and may include antibodies, antibody fragments, polypeptides, lipids, polynucleotides, and small molecules.

In one embodiment, a targeting moiety is a host viral receptor. In one aspect, the targeting moiety is a recombinant ACE-2 polypeptide. The recombinant ACE-2 may function to target the nanoparticle to the virus thereby acting as a decoy for binding of a coronavirus (e.g., a SARS-CoV-2), preventing or treating a coronavirus infection. In some embodiments, a nanoparticle composition comprising a recombinant ACE2 may prevent or treat a coronavirus infection by blocking an early stage of a coronavirus infection (e.g., a SARS-CoV-2 infection).

ACE-2 (also referred to herein as “ACE2”)(NCBI Reference Sequence: NP_068576.1) is a type I integral membrane protein and is a serine protease. It is a metallocarboxypeptidase. The active site domain of ACE-2 may be exposed to the extracellular surface of endothelial cells and the renal tubular epithelium. ACE-2 contains a 17 amino acid N-terminal signal sequence and a 22 amino acid hydrophobic transmembrane sequence near the C-terminus followed by a 43 amino acid cytoplasmic domain, which contains potential phosphorylation sites. The complete cDNA for human ACE-2 encodes a protein of 805 amino acids that exhibits 40% identity and 61% similarity to human ACE. The juxtamembrane, transmembrane and cytoplasmic domains of ACE-2 do not resemble ACE but share similarity with a 220 amino acid transmembrane glycoprotein termed collectrin, which is localized to the renal collecting ducts. Collectrin has no protease domain and its function is unknown. The homology of ACE-2 and ACE is particularly striking around the HEXXH zinc-binding motif which is identical in the two proteins. ACE-2 also contains eight cysteine residues, six of which are conserved in the N- and C-terminal domains of endothelial ACE, and has seven potential Af-linked glycosylation sites. The ACE-2 gene, located on chromosome Xp22, contains 18 exons and many of those resemble the corresponding exons in the ACE gene. ACE-2 may metabolize circulating peptides including angiotensin II, a potent vasoconstrictor and the product of angiotensin I cleavage by ACE. To this end, ACE-2 may counterbalance the effects of ACE within the renin-angiotensin system (RAS). Indeed, ACE2 has been implicated in the regulation of heart and renal function where it is proposed to control the levels of angiotensin II relative to its hypotensive metabolite, angiotensin. ACE-2 may play a unique role in the renin-angiotensin system and mediate cardiovascular and renal function.

ACE-2 may serve as a functional receptor for a coronavirus (e.g., a SARS-CoV or a SARS-CoV-2). For example, ACE-2 may facilitate respiratory tract infection of a coronavirus (e.g., SARS-CoV, SARS-CoV-2, human respiratory coronavirus NL63, or MERS-CoV). ACE-2 may be expressed in airway epithelial cells, contributing to viral infection. In some embodiments, ACE-2, but not ACE, may facilitate the association between host cells and a coronavirus S protein. Tissue distribution of ACE-2 may be consistent with the pathology of SARS-CoV. ACE-2 may be abundantly expressed in the epithelia of the lung and the small intestine, possible entry sites for a coronavirus. Nasal epithelial cells, specifically goblet/secretory cells and ciliated cells, may display the high ACE-2 expression. The skewed expression of viral receptors/entry-associated proteins towards the upper airway may be correlated with enhanced transmissivity of a viral infection. Genes associated with ACE-2 airway epithelial expression may be innate immune-associated, antiviral genes, highly enriched in the nasal epithelial cells, contributing to viral infection of airway epithelial cells.

In another embodiment, the targeting moiety may be an antibody. As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring).

In an aspect, the targeting moiety is an antibody which specifically binds a host viral receptor expressed on a cell susceptible of infection by the virus. In an exemplary embodiment, the antibody specifically binds ACE-2 expressed on the surface of epithelial cells.

### b) Components of the Composition

The present disclosure also provides pharmaceutical compositions. The pharmaceutical composition comprises a nanoparticle composition of the present disclosure, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the invention may optionally comprise one or more additional drug or therapeutically active agent in addition to the nanoparticle composition of the present disclosure. Thus, in addition to the therapies described herein, one may also provide to the subject other therapies known to be efficacious for treatment of a viral infection. In some embodiments, the secondary agent is selected from a corticosteroid, a non-steroidal anti-inflammatory drug (NSAID), an intravenous immunoglobulin, a kinase inhibitor, a fusion or recombinant protein, a monoclonal antibody, or a combination thereof. In some embodiments, agents suitable for combination therapy include but are not limited to inhaled bronchodilators and inhaled steroids.

(i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

(ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

(iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

(v) pH Modifier

In various embodiments, the excipient may be a pH modifier. By way of non-limiting example, the pH modifying agent may be sodium carbonate, sodium bicarbonate, sodium citrate, citric acid, or phosphoric acid.

(vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

In another alternate embodiment, the excipient may be a preservative. Non-limiting examples of suitable preservatives include antioxidants, such as BHA, BHT, vitamin A, vitamin C, vitamin E, or retinyl palm itate, citric acid, sodium citrate; chelators such as EDTA or EGTA; and antimicrobials, such as parabens, chlorobutanol, or phenol.

(ix) Lubricant

In a further embodiment, the excipient may be a lubricant. Non-limiting examples of suitable lubricants include minerals such as talc or silica; and fats such as vegetable stearin, magnesium stearate, or stearic acid.

(x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

(xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

A formulation comprising a composition for intranasal deliver may have a pH corresponding to a physiologically acidic nasal pH. The physiologically acidic nasal pH may depend on intact nasal mucosal function. A composition may comprise a pH of about be 6.5±0.5 (5.9 to 7.3) or about 6.7±0.6 (5.3 to 7.6). A composition may comprise a pH of about 3.8-7.7 (mean±SD 5.7±0.9). A composition for nasal deliver may be in the slightly acidic range. The average pH may have an acidity of pH 5.7.

Effective delivery of therapeutic agents via intranasal administration must take into account the decreased transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The coordinate administration methods of the instant disclosure optionally incorporate effective mucolytic or mucus-clearing agents, which serve to degrade, thin or clear mucus from intranasal mucosal surfaces to facilitate absorption and/or adsorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the invention, or as an additive within a combinatorial formulation of the invention, to provide an improved formulation that enhances intranasal delivery of biotherapeutic compounds by reducing the barrier effects of intranasal mucus.

A variety of mucolytic or mucus-clearing agents are available for incorporation within the methods and compositions of the invention. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate>taurocholate>glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein. a-amylase and β-amylase are representative of this class of enzymes, although their mucolytic effect may be limited. In contrast, bacterial glycosidases which allow these microorganisms to permeate mucus layers of their hosts.

For combinatorial use with the nanoparticles within the disclosure, non-ionogenic detergents are generally also useful as mucolytic or mucus-clearing agents. These agents typically will not modify or substantially impair the activity of the nanoparticles.

### c) Administration

(i) Dosage Forms

The composition can be formulated into various dosage forms and administered by a number of different means that will deliver a therapeutically effective amount of the active ingredient. Such compositions can be administered orally (e.g. inhalation), or parenterally in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

For parenteral administration (including subcutaneous, intraocular, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

Generally, a safe and effective amount of a nanoparticle composition is administered, for example, that amount that would cause the desired therapeutic effect in a subject while minimizing undesired side effects. In various embodiments, an effective amount of a nanoparticle composition described herein can substantially reduce viral infectivity in a subject suffering from a viral infection. In some embodiments, an effective amount is an amount capable of treating a respiratory viral infection. In some embodiments, an effective amount is an amount capable of treating one or more symptoms associated with a respiratory viral infection.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

The concentration of the nanoparticle of the present disclosure in the fluid pharmaceutical formulations can vary widely, i.e., from less than about 0.05% usually or at least about 2-10% to as much as 30 to 50% by weight and will be selected primarily by fluid volumes, viscosities, etc., in accordance with the particular mode of administration selected. For example, the concentration may be increased to lower the fluid load associated with treatment. The amount of nanoparticle pharmaceutical composition administered will depend upon the particular therapeutic entity entrapped inside the nanoparticle, the type of nanoparticle being used, and the judgment of the clinician. Generally the amount of nanoparticle pharmaceutical composition administered will be sufficient to deliver a therapeutically effective dose of the particular therapeutic entity.

The quantity of nanoparticle pharmaceutical composition necessary to deliver a therapeutically effective dose can be determined by routine in vitro and in vivo methods, common in the art of drug testing. See, for example, D. B. Budman, A. H. Calvert, E. K. Rowinsky (editors). Handbook of Anticancer Drug Development, LWW, 2003. Therapeutically effective dosages for various therapeutic entities are well known to those of skill in the art; and according to the present disclosure a therapeutic entity delivered via the pharmaceutical liposome composition of the present invention provides at least the same, or 2-fold, 4-fold, or 10-fold higher activity than the activity obtained by administering the same amount of the therapeutic entity in its routine non-liposome formulation. Typically the dosages for the nanoparticle pharmaceutical composition of the present disclosure range between about 0.005 and about 500 mg of the therapeutic entity per kilogram of body weight, most often, between about 0.1 and about 100 mg therapeutic entity/kg of body weight.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of a nanoparticle composition can occur as a single event or over a time course of treatment. For example, one or more of a nanoparticle composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a respiratory virus.

The present disclosure encompasses pharmaceutical compositions comprising compounds as disclosed above, so as to facilitate administration and promote stability of the active agent. For example, a compound of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and embodiments of the invention, the subject may be a human or any other animal.

### II. METHODS

The present disclosure encompasses methods to treat, prevent, or reduce the infectivity of a virus in a subject in need thereof. In some embodiments, the methods prevent or reduce the infectivity of a viral infection by preventing internalization of a virus into a cell of the subject or by preventing internalization of a viral genome into a cell of the subject. In some embodiments, administration of a composition provided herein, for instance those described in Section I, may disrupt or prevent an interaction between a viral surface protein (e.g., a spike protein or an envelope protein) and a host receptor protein (e.g., an epithelial angiotensin converting enzyme (ACE)). For example, administration or a nanoparticle composition may block internalization of a coronavirus into a cell of a subject by blocking or disrupting interactions between a coronavirus spike protein and a host receptor protein and/or by sequestering the virus in vivo allowing for the virus bound to the nanoparticle composition to be eliminated by the subject's immune cells. Administering a nanoparticle composition to a subject at risk for a viral infection may reduce the risk of coronavirus infection in the subject.

In some embodiments, the method include increasing ACE-2 levels in cells and tissues susceptible to infection by the virus in a subject administered a composition according to the present disclosure relative to the same cells and tissues in a subject infected by the virus and has not been administered a composition according to the disclosure. ACE-2 is down regulated in tissues including lungs of patients infected with SARS-CoV resulting in increased angiotensin 2 and acute lung injury. Therefore, the present disclosure provides methods of preventing or reducing acute lung injury in a subject infected with a respiratory virus. For example, administration of a composition as disclosed herein to a subject in need thereof increases ACE-2 levels in cells susceptible to infection by a virus thereby preventing or reducing acute lung injury in these subjects. As used herein, the phrase “cells susceptible to infection by a virus” refers to cells that express a receptor which allows the virus to infect the cell.

In some embodiments, the present disclosure provides methods to treat, prevent, or reduce the infectivity of a respiratory viral infection by targeting an anti-viral agent to cells which are susceptible to infection by the virus. Delivery of the anti-viral agent by specifically targeting cells and tissues susceptible to infection resulting in more stability, less toxicity, and targeted delivery of the anti-viral drug loaded. In some embodiments, the present disclosure provides nanoparticle compositions which functions as a nanoparticle delivery system of anti-viral drugs

In other embodiments, the present disclosure provides methods to treat, prevent, or reduce the infectivity of a respiratory viral infection. In some embodiments, the viral infection may be a coronavirus infection. The coronavirus may be SARS-CoV, SARS-CoV-2, MERS-CoV, HKU1, OC43, or 229E. The coronavirus may be a beta-coronavirus. A subject at risk for a coronavirus infection may come in contact with an asymptomatic carrier of the coronavirus infection, thereby unknowingly contracting the coronavirus infection.

In some embodiments, the compositions, methods, or treatment regiments disclosed herein may treat or prevent a SARS-CoV-2 infection (e.g., COVID-19). A SARS-CoV-2 infection may depend on host cell ACE-2 enzyme. In some embodiments, a SARS-CoV-2 infection may be blocked (e.g., prevented, treated, or slowed) by a nanoparticle composition of the disclosure. Cell entry of a coronavirus (e.g., SARS-CoV-2) may depend on binding of the viral spike (S) proteins to cellular receptors and on S protein priming by host cell proteases. For example, SARS-CoV-2 may use an ACE-2 receptor for entry. A composition of the present disclosure to treat or prevent coronavirus infection may comprise a nanoparticle comprising a host viral receptor conjugated to the nanoparticle surface, a nanoparticle comprising an antibody which specifically binds a host viral receptor. In some embodiments, the nanoparticle contains an anti-viral agent (e.g., remdesivir, chloroquine, hydroxychloroquine, lopinavir, ranitidine bismuth citrate, and ritonavir).

Generally, the methods as described herein comprise administration of a therapeutically effective amount of a nanoparticle composition of the disclosure to a subject. The methods described herein are generally performed on a subject in need thereof. A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

### III. KITS

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to compositions and pharmaceutical formulations comprising a nanoparticle composition, as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Liposome Preparation and Characterization

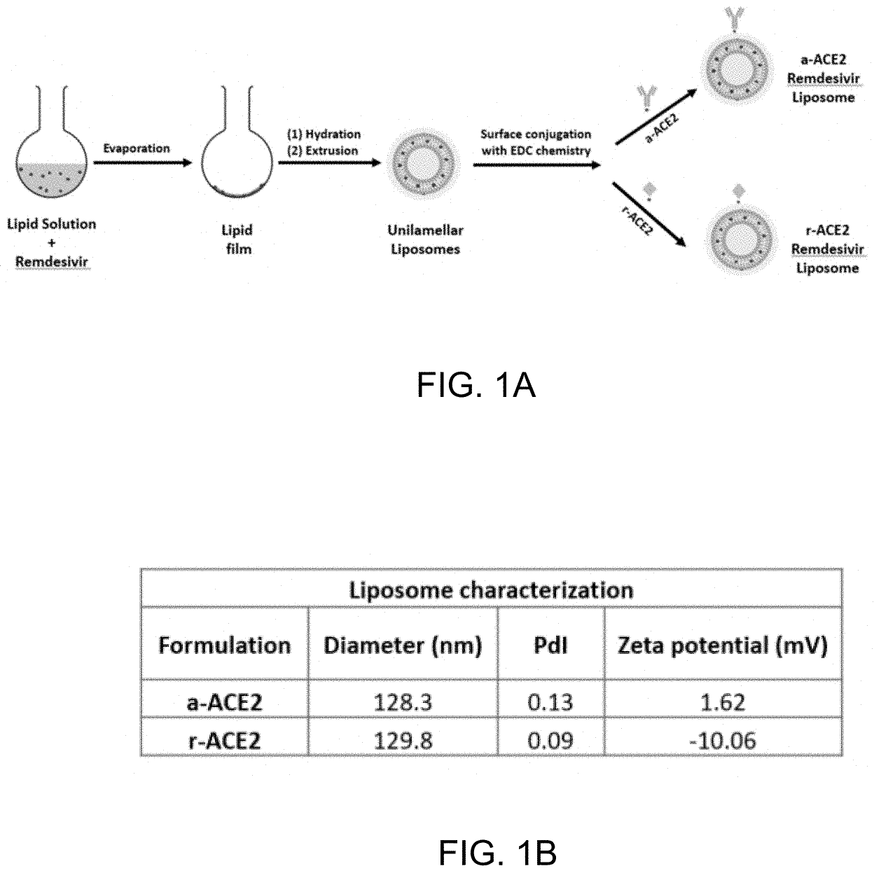

This example describes formulations of liposomal preparations. DiD (red fluorescence) liposomes containing Remdesivir were made using the thin film hydration method. Liposomes were then further conjugated with either anti-ACE2 antibody (a-ACE2) or recombinant human ACE2 protein (r-ACE2) using 1-Ethyl-3-(3-dimethylaminopropyl)carbodiimide (EDC) chemistry (FIG. 1A). Liposomes were then characterized with dynamic light scattering (DLS).

Both formulations were shown to have diameters of <130 nm, which is an adequate size shown to avoid renal clearance as well as liver/spleen accumulation. The polydispersity index (Pdl) demonstrates particle uniformity, is at an acceptable value of 0.15 or less. The zeta potential represents the surface charge of particles in a neutral buffer, and is shown to be affected by the conjugate (FIG. 1B).

An exemplary nanoparticle formulation according to the disclosure includes Formulation 1 (liposome coated with recombinant-ACE-2 and loaded with antiviral medications including but not limited to remdesivir): A liposome decorated with recombinant-ACE2 on its surface and loaded with antiviral medications including but not limited to remdesivir. This formulation will bind to the virus and will function as a “sink” to remove viral particles and at the same time deliver the loaded antiviral drug specifically to the infected cells/tissue. This formulation can be given locally (intra-nasally) or systemically (Intravenous or Intraperitoneal). Accordingly, formulation 1 functions as a “sink” for the virus. Nano-ACE-2 covered with viral particles will be eliminated by the immune cells including macrophages. Increases level of ACE-2 in infected tissues. ACE-2 is down regulated in tissues including lungs of patients infected with SARS-COV resulting in increased angiotensin 2 and acute lung injury. Increase in ACE-2 by using this formulation will prevent and help resolving acute lung injury in these patients. Functions as a nanoparticle delivery system of antiviral drugs resulting in more stability, less toxicity, and targeted delivery of the antiviral drug loaded in this formulation.

Another exemplary nanoparticle formulation according to the disclosure includes Formulation 2 (liposome coated with anti ACE-2 antibody and loaded with antiviral medications including but not limited to remdesivir): A liposome loaded with antiviral medications including but not limited to remdesivir and coated with anti-ACE2 antibodies on the surface, targeting ACE2 on the surface of epithelial cells. Formulation 2 can be given locally (intra-nasally) or systemically (Intravenous or Intraperitoneal) and carries the load of drug specifically to the ACE-2 positive cells and deliver it into the infected cells resulting in more stability, less toxicity, and targeted delivery of the antiviral drug.

Still another exemplary nanoparticle formulation according to the disclosure includes Formulation 3 (liposome coated with recombinant-ACE-2 WITHOUT antiviral loads): A liposome decorated with recombinant-ACE2 on its surface, to bind to the virus which will function as a “sink” to remove viral particles. This formulation can be given locally (intra-nasally) or systemically (intravenous or intraperitoneal) and will function as a sink for the virus. Nano-ACE-2 covered with viral particles are eliminated by the immune cells including macrophages. Increases level of ACE-2 in infected tissues. ACE-2 is down regulated in tissues including lungs of patients infected with SARS-COV resulting in increased angiotensin 2 and acute lung injury. Increase in ACE-2 by using this formulation prevent and help resolving acute lung injury in these patients.

### Example 2: In Vivo Biodistribution

DiD (red fluorescence) liposomes conjugated with a-ACE2 or r-ACE2 were subjected to biodistribution study in hamsters. Each of the formulations were administered to healthy hamsters intranasally (IN) or intraperitoneally (IP) at 2 mg per animal. The groups (n=5) are as follows: (1) a-ACE2 IN, (2) a-ACE2 IP, (3) r-ACE2 IN, (4) r-ACE2 IP.

24 hours after liposome treatment, hamsters were sacrificed. Organs were harvested, ground, and cells were analyzed using flow cytometry for DiD signal. Peripheral blood serum was isolated and measured for DiD absorbance using a spectrophotometer.

When administered IP, both formulations had minimal accumulation in the lungs, higher accumulations in the spleen and liver, as expected. When administered IN, both formulations resulted in impressive lung targeting, while significantly lowering accumulation in the spleen and liver. Additionally, a-ACE2 IN formulation specifically targeted to the lungs while avoiding other organs, including intestines. r-ACE2 IN formulation had high intestine accumulation, however, it could be due to swallowing during the administration process.

Looking at liposomes remaining in the blood, IP formulations had much higher levels at 24 hours compared to IN formulations. Combined with what was observed with organ biodistribution, IP formulations infer higher side effect profiles then IN formulations.

### Example 3: Prohylaxis and Treatment of SARS-CoV-2 Animal Models

Nanoparticle formulations according to the disclosure were administered before inoculation (prevention) or after inoculation (treatment) of the animals with SARS-CoV-2. Animals were monitored for signs of infection (e.g, weight and respiratory rate, and death) and viral load in various organs including lungs was determined.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

1. A method of reducing or treating a respiratory viral infection in a subject, the method comprising:
administering to the subject a pharmaceutical composition comprising at least one pharmaceutically acceptable excipient and a nanoparticle comprising at least one targeting moiety conjugated to the surface and wherein the targeting moiety is selected from a recombinant angiotensin converting enzyme-2 (ACE-2) polypeptide and an anti-ACE-2 antibody.

2. The method of claim 1, wherein the nanoparticle is a liposome.

3. The method of claim 1, wherein the nanoparticle further comprises an anti-viral agent.

4. The method of claim 3, wherein the anti-viral agent is selected from remdesivir, chloroquine, hydroxychloroquine, lopinavir, ranitidine bismuth citrate, and ritonavir.

5. The method of claim 1, comprising nasally administering the pharmaceutical composition to the subject.

6. The method of claim 1, wherein the respiratory virus is a coronavirus.

7.-9. (canceled)

10. The method of claim 1, wherein infectivity of the virus is reduced by disrupting or preventing an interaction between a viral surface protein and a host receptor protein.

11. The method of claim 10, wherein the viral surface protein is a spike protein and the host receptor protein is ACE-2.

12. The method of claim 1, wherein levels of ACE-2 are is-increased in cells or tissues susceptible to infection by the virus in the subject relative to cells and tissues of a subject infected by the virus and not administered the pharmaceutical composition.

13. The method of claim 12, wherein acute lung injury is prevented or reduced.

14. (canceled)

15. A pharmaceutical composition comprising at least one pharmaceutically acceptable excipient and a nanoparticle comprising at least one targeting moiety conjugated to the surface, wherein the targeting moiety is a recombinant angiotensin converting enzyme-2 (ACE-2) polypeptide.

16. The pharmaceutical composition of claim 15, wherein the nanoparticle is a liposome.

17. The pharmaceutical composition of claim 15, wherein the nanoparticle further comprises an anti-viral agent.

18. The pharmaceutical composition of claim 17, wherein the anti-viral agent is selected from remdesivir, chloroquine, hydroxychloroquine, lopinavir, ranitidine bismuth citrate, and ritonavir.

19. The pharmaceutical composition of claim 15, wherein the pharmaceutical composition is formulated for nasal delivery.

20. (canceled)

21. A pharmaceutical composition comprising at least one pharmaceutically acceptable excipient and a nanoparticle comprising at least one targeting moiety conjugated to the surface, wherein the targeting moiety is an anti-ACE-2 antibody.

22. The pharmaceutical composition of claim 21, wherein the nanoparticle is a liposome.

23. The pharmaceutical composition of claim 21, wherein the nanoparticle further comprises an anti-viral agent.

24. The pharmaceutical composition of claim 23, wherein the anti-viral agent is selected from remdesivir, chloroquine, hydroxychloroquine, lopinavir, ranitidine bismuth citrate, and ritonavir.

25. The pharmaceutical composition of claim, wherein the pharmaceutical composition is formulated for nasal delivery.

26. (canceled)
